(19) United States
(12) Patent Application Publication
(10) Pub. No.: US 2012/0201901 A1
(43) Pub. Date: Aug. 9, 2012

(54) Topical Antibiotic Formulations

(75) Inventors: Perry Antelman, Sharon, MA (US); David Goldsmith, Harmony, RI (US); Shalom Lampert, Maalot (IL)

(73) Assignee: AIDANCE SKINCARE & TOPICAL SOLUTIONS, LLC, Woonsocket, RI (US)

(21) Appl. No.: 13/021,755

(22) Filed: Feb. 6, 2011

(30) Foreign Application Priority Data
Feb. 4, 2011 (GB) GB1101936.1

(51) Int. Cl. A61K 33/38; A61K 38/12; A61K 31/7036; A61K 31/7048; A61P 31/00; A61K 47/14; A61K 47/46; A61K 47/02; A61K 47/36; A61K 31/555; A61K 31/165

(52) U.S. Cl. 424/618; 514/184; 514/2.3; 514/39; 514/29; 514/628; 514/785; 514/783; 514/770; 514/778

(57) ABSTRACT

A biocompatible putty formulation suitable for insertion within chronic and acute wounds of humans and animals, the formulation containing a topical antibiotic, a biocompatible humectant, a biocompatible viscosity-building agent, and at least 5% water, by weight, the humectant and the viscosity-building agent intimately mixed within the formulation, the formulation adapted whereby the formulation remains a solid over the entire temperature range of 20° C. to 35° C.

### CROSS-REFERENCE TO RELATED APPLICATIONS

This application draws priority from UK Patent Application No. GB1101936.1, filed Feb. 4, 2011.

### FIELD AND BACKGROUND OF THE INVENTION

The present invention relates to topical, antibiotic formulations, and in particular, to topical, antibiotic formulations for treating chronic and acute wounds.

Chronic wound care is a critical and growing issue in healthcare systems. A chronic wound may be defined as a wound that shows no sign of appreciable healing within 2-3 months. Chronic wounds such as skin ulcers are the most common complication of diabetes, which has been termed a “Silent Epidemic”. Above and beyond their economic burden on healthcare systems, chronic wounds represent a debilitating problem having significant clinical and social ramifications. Chronic wounds may be non-responsive or poorly responsive to various known treatments. Consequently, such chronic wounds may become severely infected, leading to gangrene and amputations.

While some advances have been made in the treatment of wounds, both chronic and acute, we believe there is a need for further improvements in formulating stable, efficacious topical antibiotic formulations and medical devices, and the subject matter of the present disclosure and claims is aimed at fulfilling this need.

### SUMMARY OF THE INVENTION

According to the teachings of the present invention there is provided a solid biocompatible formulation suitable for insertion within chronic and acute wounds of humans and animals, the formulation including a topical antibiotic, a biocompatible humectant, and a biocompatible viscosity-building agent, the humectant and the viscosity-building agent intimately mixed within the formulation, which is formulated and adapted whereby the formulation remains a solid over at least an entire temperature range of 20° C. to 35° C., the solid formulation having a storage modulus (G′) and a loss modulus (G″), both measured at 25° C. and within a frequency range of 0.1 Hz to 1.0 Hz, and a complex modulus (G*), defined by:

G*=(G′²+G″²)^{1/2}

the formulation having at least one of the following five rheological properties:

(1) in a torque sweep at a frequency of 1.0 Hz, the complex modulus achieves a plateau or a maximum of at least 4.0×10⁴ Pa, at least 6.0×10⁴ Pa, at least 8.0×10⁴ Pa, or at least 10.0×10⁴ Pa;

(2) in the torque sweep, the complex modulus drops sharply, or begins to exhibit non-linear behavior, at an oscillating stress of at least 800 Pa, at least 900 Pa, at least 1000 Pa, at least 1200 Pa, at least 1500 Pa, or at least 2000 Pa;

within the frequency range, at least one point:

(3) the storage modulus is at least 1.0×10⁴ Pa, at least 2.0×10⁴ Pa, at least 3.0×10⁴ Pa, at least 4.0×10⁴ Pa, at least 5.0×10⁴ Pa, or at least 6.0×10⁴ Pa;

(4) the loss modulus is at least 0.4×10⁴ Pa, at least 0.5×10⁴ Pa, at least 0.6×10⁴ Pa, at least 0.8×10⁴ Pa, at least 1.0×10⁴ Pa, at least 1.5×10⁴ Pa or at least 2.0×10⁴ Pa;

(5) the complex modulus is at least 1.05×10⁴ Pa, at least 1.05×10⁴ Pa, at least 2×10⁴ Pa, at least 3.0×10⁴ Pa, at least 4.0×10⁴ Pa, or at least 6.0×10⁴ Pa.

According to further features in the described preferred embodiments, the complex modulus achieves a plateau or maximum of at least 4.0×10⁴ Pa, at least 6.0×10⁴ Pa, at least 8.0×10⁴ Pa, or at least 10.0×10⁴ Pa.

According to still further features in the described preferred embodiments, the complex modulus drops sharply, or begins to exhibit non-linear behavior, at an oscillating stress of at least 800 Pa, at least 900 Pa, at least 1000 Pa, at least 1200 Pa, at least 1500 Pa, or at least 2000 Pa.

According to still further features in the described preferred embodiments, at least one point within the frequency range, the storage modulus is less than 1.2×10⁷ Pa, less than 1.0×10⁷ Pa, less than 8×10⁶ Pa, or less than 7×10⁶ Pa.

According to still further features in the described preferred embodiments, at least one point within the frequency range, the loss modulus is less than 5×10⁶ Pa, less than 3×10⁶ Pa, less than 2×10⁶ Pa, or less than 1×10⁶ Pa.

According to still further features in the described preferred embodiments, at least one point within the frequency range, the complex modulus is less than 1.2×10⁷ Pa, less than 1.0×10⁷ Pa, less than 8×10⁶ Pa, or less than 7×10⁶ Pa.

According to still further features in the described preferred embodiments, at least one point within the frequency range, a ratio of the storage modulus to the loss modulus is at least 1.5:1, at least 2.0:1, at least 2.5:1, at least 3:1, at least 4:1, or at least 5:1, and/or the ratio is less than 12:1, less than 10:1, less than 9:1, or less than 8:1.

According to still further features in the described preferred embodiments, at least one point within the frequency range, the storage modulus is at least 3.0×10⁴ Pa, at least 4.0×10⁴ Pa, at least 5.0×10⁴ Pa, or at least 6.0×10⁴ Pa, and the loss modulus is at least 0.6×10⁴ Pa, at least 0.8×10⁴ Pa, at least 1.0×10⁴ Pa, at least 1.5×10⁴ Pa, or at least 2.0×10⁴ Pa.

According to still further features in the described preferred embodiments, at least one point within the frequency range, the storage modulus is at least 5.0×10⁴ Pa, or at least 6.0×10⁴ Pa, and the loss modulus is at least 0.8×10⁴ Pa, at least 1.0×10⁴ Pa, at least 1.5×10⁴ Pa, or at least 2.0×10⁴ Pa.

According to still further features in the described preferred embodiments, a ratio of the storage modulus to the loss modulus is at least 1.5:1, at least 2.0:1, at least 2.5:1, at least 3:1, at least 4:1, or at least 5:1, and/or the ratio is less than 12:1, less than 10:1, less than 9:1, or less than 8:1, substantially throughout the frequency range.

According to still further features in the described preferred embodiments, the storage modulus is at least 3.0×10⁴ Pa, at least 4.0×10⁴ Pa, at least 5.0×10⁴ Pa, or at least 6.0×10⁴ Pa, substantially throughout the frequency range.

According to still further features in the described preferred embodiments, the loss modulus is at least 0.6×10⁴ Pa, at least 0.8×10⁴ Pa, at least 1.0×10⁴ Pa, at least 1.5×10⁴ Pa, or at least 2.0×10⁴ Pa, substantially throughout the frequency range.

According to still further features in the described preferred embodiments, the storage modulus is at least 5.0×10⁴ Pa, or at least 6.0×10⁴ Pa, substantially throughout the frequency range.

According to still further features in the described preferred embodiments, the loss modulus is at least 1.0×10⁴ Pa, at least 1.5×10⁴ Pa, or at least 2.0×10⁴ Pa, substantially throughout the frequency range.

According to still further features in the described preferred embodiments, the water concentration within the formulation is at least 5%, at least 7%, at least 10%, at least 20%, at least 30%, at least 40%, at least 50%, or at least 60%.

According to still further features in the described preferred embodiments, the concentration of the antibiotic within the formulation is at least 0.1%, at least 0.2%, at least 0.4%, at least 0.7%, or at least 1%.

According to still further features in the described preferred embodiments, the antibiotic is present within the formulation in a therapeutically effective concentration for treatment of topical skin infections.

According to still further features in the described preferred embodiments, the antibiotic is selected from the group of topical antibiotics consisting of silver(II) oxide, silver(I) oxide, silver sulfadiazine, Bacitracin, Neomycin, Erythromycin and Chloramphenicol.

According to still further features in the described preferred embodiments, the antibiotic consists largely of, predominantly of, or substantially of, the silver(II) oxide.

According to still further features in the described preferred embodiments, the formulation contains, by weight, at least 0.10%, at least 0.20%, at least 0.30%, or at least 0.50%, silver(I) oxide and/or silver(II) oxide.

According to still further features in the described preferred embodiments, the humectant and the viscosity-building agent are selected, and the formulation is adapted, whereby a melting temperature of the formulation is at least 40° C., at least 45° C., at least 50° C., or at least 75° C.

According to still further features in the described preferred embodiments, the formulation is a putty at 20° C. or at 22° C.

According to still further features in the described preferred embodiments, the formulation is a putty at 35° C. or at 37° C.

According to still further features in the described preferred embodiments, the formulation is a putty throughout the temperature range.

According to still further features in the described preferred embodiments, the formulation contains at least 1%, at least 1.5%, at least 2.5%, at least 3%, at least 4%, at least 7%, at least 12%, at least 20%, or at least 30% of the humectant.

According to still further features in the described preferred embodiments, the formulation contains less than about 55%, less than 50%, less than 48%, less than 45%, or less than 40%, of the humectant.

According to still further features in the described preferred embodiments, the humectant includes, largely includes, predominantly includes, or consists essentially of a liquid wax ester.

According to still further features in the described preferred embodiments, the formulation further includes an absorbefacient.

According to still further features in the described preferred embodiments, the formulation further includes an absorbefacient, wherein a combined weight content of the viscosity-building agent and the absorbefacient within the formulation is at least about 4%, at least 6%, at least 8%, at least 10%, or at least 15%.

According to still further features in the described preferred embodiments, the combined weight content is in a range of about 8% to 70%, about 8% to 65%, or about 10% to 50%.

According to still further features in the described preferred embodiments, the viscosity-building agent includes, largely includes, or consists essentially of at least one of a hydrophyllic clay, a flour, and a starch.

According to still further features in the described preferred embodiments, the absorbefacient includes, largely includes, or consists essentially of at least one of a hydrophyllic clay, a flour, and a starch.

According to still further features in the described preferred embodiments, the hydrophyllic clay is selected from at least one of the group of hydrophyllic clays consisting of a smectite, sepiolite, and palygorskite.

According to still further features in the described preferred embodiments, the smectite is selected from at least one of the group consisting of bentonite, montmorillonite and hectorite.

According to still further features in the described preferred embodiments, a weight ratio of the at least one viscosity-building agent and absorbefacient to humectant is at least 0.25:1, at least 0.4:1, at least 0.6:1, at least 1:1, and more typically, about 1.5:1 to 5:1, about 2:1 to 5:1, or about 2:1 to 4:1.

According to still further features in the described preferred embodiments, the humectant includes jojoba oil, hydrogenated jojoba oil.

According to still further features in the described preferred embodiments, the humectant includes, largely includes, or consists essentially of jojoba oil.

According to still further features in the described preferred embodiments, the formulation further includes at least 0.3%, at least 1%, at least 2.5%, or at least 4% of a skin-protecting agent.

According to still further features in the described preferred embodiments, the skin-protecting agent includes zinc oxide.

According to still further features in the described preferred embodiments, the formulation contains, by weight, less than 15%, less than 12%, or less than 10% of the skin-protecting agent.

According to still further features in the described preferred embodiments, the formulation is an elastic, moldable formulation.

According to still further features in the described preferred embodiments, the formulation is adapted whereby a plug or piece of the formulation may be fit to a contour of a wound cavity

According to still further features in the described preferred embodiments, the formulation is adapted whereby a plug or piece of the formulation may be inserted into a wound cavity in an integral fashion.

According to still further features in the described preferred embodiments, the formulation is adapted wherein a plug or piece of the formulation securely holds position within a wound cavity.

According to still further features in the described preferred embodiments, the formulation and/or a plug or piece of the formulation is adapted to be removed from a wound cavity in an integral fashion.

According to still further features in the described preferred embodiments, the formulation and/or a plug or piece of the formulation is adapted to provide a gentle pressure against a surface within a wound cavity.

According to still further features in the described preferred embodiments, the formulation and/or a plug or piece of the formulation is adapted to be removed from the wound cavity in an integral fashion, after contacting the surface for at least 4 hours, at least 12 hours, or at least 24 hours.

According to yet another aspect of the present invention there is provided a formulation suitable for application to skin tissue, substantially as described herein, the formulation including any feature described, either individually or in combination with any feature, in any configuration.

According to another aspect of the present invention there is provided a method of producing a composition, formulation, or medical device, the method including any feature described, either individually or in combination with any feature, in any configuration.

According to another aspect of the present invention there is provided a method of topically applying the inventive formulation or medical device on the skin, on a wound, or within a wound cavity, the method comprising any feature described, either individually or in combination with any feature, in any configuration.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 provides a plot of the storage modulus G′ and the loss modulus G″, as a function of frequency, for a first formulation of the present invention;

FIG. 2 provides a plot of the storage modulus G′ and the loss modulus G″, as a function of frequency, for a second formulation of the present invention;

FIG. 3 shows a torque sweep as a function of the oscillating stress, for a third formulation of the present invention;

FIG. 4 provides a plot of the storage modulus G′ and the loss modulus G″, as a function of frequency, for the formulation of FIG. 3;

FIG. 5 provides bar graphs of the zones of inhibition of various formulation of the present invention; and

FIG. 6 provides bar graphs showing clinical wound closure data from comparative clinical trials in which the use of an exemplary putty of the present invention is tested against the use of a silver oxide ointment and against a conventional treatment protocol.

### DESCRIPTION OF THE PREFERRED EMBODIMENTS

Before explaining at least one embodiment of the invention in detail, it is to be understood that the invention is not limited in its application to the details set forth in the following description. The invention may be capable of other embodiments or of being practiced or carried out in various ways. Also, it is to be understood that the phraseology and terminology employed herein is for the purpose of description and should not be regarded as limiting.

A first aspect of the present invention relates to a solid or substantially solid formulation or medical device, typically having a putty-like consistency, which may be particularly efficacious as a topical antibiotic in various applications. Such formulations or medical devices may exhibit superior oxidative stability and superior phase stability, along with efficacy in the inhibition, treatment and cure of various dermatological conditions. This formulation may be particularly efficacious in the treatment of bedsores, diabetic ulcers such as diabetic foot ulcers, puncture wounds, and the like.

The inventive putty formulation may include at least one viscosity-building agent, typically including a hydrophyllic clay or smectite such as bentonite or hectorite, or an organoclay such as a bentonite or hectorite organoclay, a humectant, typically including an oil or liquid wax ester such as jojoba oil, and a base liquid, typically water or an aqueous solvent. The formulation may advantageously include an absorbefacient.

The viscosity-building agent may include, largely include, predominantly include, or consist essentially of a flour (such as wheat flour, corn flour, and/or rice flour) and/or a starch (such as corn starch or potato starch).

The formulation may advantageously include, in addition to an antibiotic agent, at least one preservative adapted to inhibit bacterial and/or fungal growth within the putty formulation. Preferably, the preservative, or combination of preservatives, should be effective against bacteria, molds and yeasts. Such preservatives may include at least one of benzoic acid, salicylic acid, and various parabens. While various preservatives are known to those of ordinary skill in the art of cosmetic and pharmaceutical formulations, it will be appreciated that the chemical compatibility with silver(II) and silver(I) oxide must be tested, for those formulations containing such silver oxides.

Preferred antibiotics may include at least one silver oxide. Preferably, the inventive solid or substantially solid formulation may include a silver(II) oxide such as tetrasilver tetroxide, or a silver(I) oxide such as Ag₂O or silver sulfadiazine. To benefit from the bacteriostatic and antibiotic properties of the silver(II) oxide, the formulation may contain, by weight, at least 0.025% of the silver(II) oxide, and more typically, at least 0.05%, at least 0.10%, at least 0.25%, or 0.25% to 3.5 or 4% thereof. To benefit from the bacteriostatic and bacteriocidal properties of the silver(I) oxide, the formulation may contain, by weight, at least 0.05% of the silver(I) oxide, and more typically, at least 0.10%, at least 0.25%, or 0.25% to 3.5% thereof.

In topical applications such as the treatment of chronic wounds and acute wounds, the inventive formulation preferably exhibits particular mechanical, physical, bacteriocidal, palliative, moisturizing, and skin-protecting or skin-building properties. It is also essential that the various components of the formulation are biocompatible and are compatible with one another.

In some applications, it may be essential for the inventive formulation to be highly absorbefacient, in order to dry up fluid serving as a medium for microbial growth. However, we have found that a delicate balance may exist between inducing absorption and moisturization. Without a suitable moisturization agent or means, the absorption process may disadvantageously dry up the surrounding tissue, which may promote tissue irritation and skin cracking and induce pain, discomfort, and even additional infection. Moreover, we have found that the activity of various antibiotic agents (e.g., silver(II) oxide) may be compromised in dry environments, further constraining the balance between formulation absorption and moisturization.

To this end, we have found that the putty or plaster formulation of the present invention may advantageously include at least about 1%, at least about 1.5%, at least about 2.5%, at least about 3%, at least about 4%, and preferably, about 4% to 55%, about 4% to 50%, about 4% to 45%, about 5% to 40%, about 5% to 30%, or about 5% to 20%, by weight, of a humectant such as a liquid wax ester and/or an oil. The humectant may typically include, largely conclude, or consist mainly or predominantly of, a liquid wax ester such as jojoba oil. Additional humectants will be readily apparent to those of ordinary skill in the art.

The humectant may serve to mitigate or otherwise counter the drying effect of the absorbefacient. At higher concentrations of humectant, the humectant may leak out, ooze out, or be otherwise discharged from the formulation, making the use of the formulation less clean and convenient for medical practitioners and he patient.

Typically, the putty formulation may include at least about 2%, at least about 5%, at least about 8%, at least about 12%, or at least about 20%, by weight, and preferably, about 2% to 50%, about 3% to 45%, or about 4% to 40%, by weight, of at least one such absorbefacient. In these concentrations, the absorbefacient may serve a dual function as a viscosity-building agent. Various phyllosilicates or clays, including smectites, sepiolite and palygorskite, or organoclays such as disteardimonium bentonite may advantageously behave both as an absorbefacient and as a viscosity-building agent. The smectite may include various natural and synthetic forms of bentonite, montmorillonite and hectorite. It may be appreciated by one of skill in the art that hectorite may be somewhat more potent than bentonite and montmorillonite as an absorbefacient and as a viscosity-building agent, on a per-weight basis, such that lower concentrations of hectorite may be used to achieve the desired results. Those of ordinary skill in the art may readily identify other absorbefacient substances that may be suitable for use in the formulations according to the present invention.

It must be emphasized that the inventive formulation may be therapeutically effective in the treatment of wounds and skin infections, even without an antibiotic agent. Without wishing to be bound by theory, the inventors believe that the absorbefacient nature of the formulation is efficacious in reducing the moisture within the wound cavity, negatively impacting the growth environment of the microorganisms.

The inventive putty formulation may further include a skin-protecting or skin-building agent. Typically, the formulation may advantageously include at least 0.2%, and more typically, 1% to 15% or 2% to 10%, by weight, of the skin-protecting or skin-building agent. One presently preferred agent is zinc oxide.

The solvent typically includes water. Water may constitute at least 2%, at least 5%, at least 10%, at least 25%, at least 35%, or at least 40%, by weight, of the inventive formulation, and more typically, about 40 or 45% to 75%, or about 50% to 70% thereof.

We have discovered that with regard to various formulations of the present invention, a high weight ratio of the smectite (or more generally of the total weight of the at least one viscosity-building agent and absorbefacient) to the at least one antibiotic (e.g., Ag₂O, a silver(II) oxide such as tetrasilver tetroxide, or Bacitracin, Neomycin and the like) may not reduce the anti-microbial efficacy of the formulation. Weight ratios of up to 600:1 (smectite to antibiotic such as silver(II) oxide), up to 250:1, up to 100:1, up to 50:1, or up to 25:1 may display no decrease in anti-microbial efficacy (relative to substantially identical formulations having no smectite content) with respect to various skin-related microorganisms.

In many formulations of the present invention, the weight ratio of the smectite (or more generally of the total weight of the at least one viscosity-building agent and absorbefacient) to the at least one antibiotic is at least 0.2:1, at least 0.5:1, at least 1:1, at least 2:1, at least 5:1, at least 10:1, at least 20:1, or at least 50:1.

Bentonite, montmorillonite and hectorite are presently preferred smectites.

With particular regard to the putty formulations (including thick, viscous plaster formulations) of the present invention, the putty formulation may have a weight ratio of at least one viscosity-building agent and absorbefacient (e.g., a smectite) to the at least one antibiotic (e.g., silver(II) oxide) of at least 5:1, and more typically, about 5:1 to 200:1, about 5:1 to 75:1, or about 10:1 to 60:1.

In the putty formulation of the present invention, the weight ratio of the at least one viscosity-building agent and absorbefacient to at least one humectant (e.g., jojoba oil) may be at least 0.25:1, at least 0.4:1, at least 0.6:1, at least 1:1, and more typically, about 1.5:1 to 5:1, about 2:1 to 5:1, or about 2:1 to 4:1. The inventive putty formulation may have various rheological properties that are particularly suited to various topical applications. For example, the putty may have an overall flexibility that is sufficient to enable molding of the putty to conform or largely conform to the shape of various surfaces. For example, a cavity of a wound or bedsore may be filled or partially filled with the inventive putty, whereby the putty conforms to the shape of the cavity. The putty may be inserted into the wound cavity as an integral piece, or as integral pieces. The putty may exhibit sufficient rigidity or stiffness to maintain its position over time (e.g., at least 1 hour, at least 2 hours, at least 4-12 hours, at least 24 hours, at least 48 hours, or at least 72 hours), within such a cavity, without oozing out, falling out, etc. The putty may exhibit sufficient rigidity or stiffness even as the temperature of the putty increases from room temperature to the temperature within the wound of the patient (human or animal).

The inventive putty formulation may advantageously be adapted to retain its integrity within the wound cavity, whereby the putty may be removed as an integral piece after at least 1 hour, at least 2 hours, at least 4 hours, or even after at least 24-72 hours.

The putty formulation may be rheologically adapted to apply a gentle and/or constant pressure against the surrounding tissue. While such pressure contact may promote improved contact between the antibiotic agent and the microorganisms, the contact may, in medical devices and techniques of the prior art, result in sticking of the medical device (e.g., gauze) to the wound surface. Absorbefacients pressure-contacted with a wound surface may excessively dry out the surface. Such effects may adversely affect wound healing, and may subject the patient to discomfort or acute pain. Absorbefacients pressure-contacted with the wound surface may also disintegrate or stick to the wound surface.

By sharp contrast, the inventive formulation may be adapted to remain integral within the wound cavity, to pressure-contact the wound surfaces without sticking thereto, and to be removed with facility from the wound. The formulation may be loaded with sufficient humectant, whereby excessive drying out of the wound surface is avoided, even over several days of continuous presence within the wound cavity.

Various rheological properties of the inventive putty formulation, such as viscosity and/or complex modulus (G*), may be generally maintained between room temperature (about 20-22° C.) and body temperature (about 32-35° C.). This may not be true for various materials or carriers based on petroleum, by way of example. Thus, the formulation components may be selected, and the formulation may be prepared, whereby the melting temperature of the formulation as a whole, is at least 40° C., at least 45° C., at least 50° C., or more typically, at least 75° C.

In characterizing the rheological properties of the present invention, we have found that the inventive formulation may have a large storage modulus (G′) relative to the loss modulus (G″). Using a rotational rheometer such as a TA Instruments G2 rotational rheometer, we have found that the storage modulus, at any point or at substantially every point in the frequency range of 0.1 Hz to 1.0 Hz, may be at least 0.2×10⁴ Pa, at least 0.5×10⁴ Pa, at least 1.0×10⁴ Pa, at least 2×10⁴ Pa, at least 3.0×10⁴ Pa, at least 4.0×10⁴ Pa, at least 6.0×10⁴ Pa, at least 9.0×10⁴ Pa, or at least 12.0×10⁴ Pa. At any point or at substantially every point in this frequency range, the storage modulus may be less than 1.2×10⁷ Pa, less than 1.0×10⁷ Pa, less than 8×10⁶ Pa, or less than 7×10⁶ Pa. More typically, the storage modulus may be within a range of 3.0×10⁴ Pa to 1.0×10⁷ Pa, within a range of 3.5×10⁴ Pa to 9×10⁶ Pa, within a range of 4.0×10⁴ Pa to 7×10⁶ Pa, or within a range of 5.0×10⁴ Pa to 7×10⁶ Pa.

In further characterizing these structural rheological properties, we have found that, at any point or at substantially every point in the frequency range of 0.1 Hz to 1.0 Hz, the loss modulus of the inventive putty formulation may be at least 0.1×10⁴ Pa, at least 0.4×10⁴ Pa, at least 0.5×10⁴ Pa, at least 0.6×10⁴ Pa, at least 0.8×10⁴ Pa, or at least 1.0×10⁴ Pa. At any point or at substantially every point in this frequency range, the loss modulus may be less than 5×10⁶ Pa, less than 3×10⁶ Pa, less than 2×10⁶ Pa, or less than 1×10⁶ Pa.

The ratio of the storage modulus to the loss modulus, at any point or at substantially every point in the frequency range of 0.1 Hz to 1.0 Hz, may be at least 1.0:1, at least 1.5:1, at least 2.0:1, at least 2.5:1, at least 3:1, at least 4:1, or at least 5:1. This ratio may be less than 12:1, less than 10:1, less than 9:1, or less than 8:1. The ratio of the storage modulus to the loss modulus may be in a range of 2.5:1 to 12:1, 3:1 to 10:1, or 4:1 to 9:1. Some formulations of the present invention have a storage modulus to loss modulus ratio of 4.5:1 to 7.5:1, or 5:1 to 7:1.

The complex modulus (G*), which is defined by the equation:

G*=(G′²+G″²)^{1/2}

may be, at any point or at substantially every point in this frequency range, at least 0.3×10⁴ Pa, at least 0.5×10⁴ Pa, at least 0.7×10⁴ Pa, at least 1.0×10⁴ Pa, at least 2×10⁴ Pa, at least 3.0×10⁴ Pa, or at least 4.0×10⁴ Pa, at least 6.0×10⁴ Pa, at least 9.0×10⁴ Pa, at least 12.0×10⁴ Pa, or at least 12.0×10⁴ Pa. At any point or at substantially every point in this frequency range, the complex modulus may be less than 1.2×10⁷ Pa, less than 1.0×10⁷ Pa, less than 8×10⁶ Pa, or less than 7×10⁶ Pa. More typically, the complex modulus may be within a range of 1.0×10⁴ Pa to 1.0×10⁷ Pa, within a range of 2.0×10⁴ Pa to 1.0×10⁷ Pa, within a range of 3.0×10⁴ Pa to 1.0×10⁷ Pa, within a range of 3.5×10⁴ Pa to 9×10⁶ Pa, or within a range of 4.0×10⁴ Pa to 7×10⁶ Pa.

At least one point within the frequency range, the ratio of the storage modulus to the loss modulus is at least 1.5:1, at least 2.0:1, at least 2.5:1, at least 3:1, at least 4:1, or at least 5:1, and/or the ratio is less than 12:1, less than 10:1, less than 9:1, or less than 8:1.

### EXAMPLES

Reference is now made to the following examples, which together with the above description, illustrate the invention in a non-limiting fashion.

### Example 1

To a container containing water or more generally, an aqueous medium, is added at least one viscosity-building agent, typically a smectite (e.g., a bentonite or montmorillonite powder such as Gelwhite H, produced by Southern Clay Products, Inc., Gonzales, Tex.). The mixture is vigorously mixed or homogenized, typically for 0.5 to 2 hours, typically producing a single, viscous phase. The phase is typically homogeneous or substantially homogeneous. The oil and/or liquid wax ester (e.g., jojoba oil) may be introduced to the mixture during the mixing (e.g., blending or homogenizing), typically after the viscosity has been built. Mixing may be continued as the antibiotic (e.g., tetrasilver tetroxide) and various optional ingredients (e.g., skin builders) are introduced. Further mixing may ensue, typically for 5-30 minutes. Viscosity-building agents such as flours and starches may be introduced towards the end of the preparation process; a dough hook may advantageously be used for the subsequent mixing. The viscous formulation is typically homogeneous or substantially homogeneous.

### Example 2

A putty formulation was prepared according to the procedure provided in Example 1. The putty contained included approximately 66% water, 24% bentonite, 9% jojoba oil, and 0.88% tetrasilver tetroxide.

### Example 3

A putty formulation was prepared according to the procedure provided in Example 1. The putty contained included approximately 65% water, 23% bentonite, 9% jojoba oil, 2% zinc oxide, and 0.88% tetrasilver tetroxide.

### Example 4

A putty formulation was prepared according to the procedure provided in Example 1. The putty contained approximately 53% water, 37% bentonite, 9% jojoba oil, and 0.88% tetrasilver tetroxide.

### Example 5

A putty formulation was prepared according to the procedure provided in Example 1. The putty contained 53% water, 35% bentonite, 9% jojoba oil, 2% zinc oxide, and 0.88% tetrasilver tetroxide.

### Example 6

A putty formulation was prepared according to the procedure provided in Example 1. The putty contained approximately 41% water, 3.4% bentonite, 14.2% jojoba oil, 41% flour, and 0.5% tetrasilver tetroxide. The putty was highly pliable and exhibited excellent phase stability.

### Example 7

A putty formulation was prepared according to the procedure provided in Example 1. The putty contained approximately 47% water, 10% bentonite, 43% jojoba oil, and under 0.1% tetrasilver tetroxide. The putty formulation was designated as Sample 11010-1.

### Example 8

A putty formulation was prepared according to the procedure provided in Example 1. The putty contained approximately 46% water, 34% bentonite, 13% jojoba oil, 5% zinc oxide, and 1% tetrasilver tetroxide. The putty formulation was designated as Sample 11010-2.

### Examples 9-10

The putty formulations of Example 7 and Example 8 were subjected to rheological evaluation using a TA Instruments G2 rotational rheometer.

Small amplitude oscillatory rheometry was conducted on the provided samples, using a two-centimeter, stainless steel parallel plate geometry. To overcome sample-loading issues, the two samples were placed on the Peltier plate of the rheometer, and partially flatted with a flat Teflon® plate. Two one-millimeter shims were placed on either side of the sample, and a doctor blade was used to trim the sample to approximately 1000 micrometers. The two-centimeter parallel plate was then lowered onto the sample, achieving a gap distance between 1000 and 1050 micrometers.

The samples were initially subjected to a stress sweep at 1 Hz to identify the suitable oscillating torque for the frequency sweep based on waveform shape and the onset of non-linear viscoelastic behavior. Based on this work, a torque of 1000 μN-m was used for 11010-1, and 8000 μN-m for 11010-2.

Frequency sweeps were then conducted on both samples from 0.01 to 100 Hz at 25° C. and the indicated oscillating torques. Ten points were collected per decade of oscillating frequency. The run for sample 11010-1 showed inertial effects at frequencies above 16 Hz; as such, the data curve was truncated.

Both samples exhibit strongly elastic behavior in the regime tested, indicated by a large storage modulus G′ relative to the loss modulus G″. Sample 11010-1 is substantially less stiff than 11010-2 by approximately a factor of 60-70. At 1 Hz, sample 11010-1 had a storage modulus of 6.26×10⁴ Pa, while sample 11010-2 had a storage modulus of 4.32×10⁶ Pa. Both samples show a modest onset of a terminal zone at a frequency of approximately 0.03 Hz, followed by a quasi-plateau modulus. No strain hardening was observed in the achievable upper range of frequencies tested.

The complex modulus, G*, is the resultant vector of the storage and loss modulus. A higher complex or overall modulus indicates a stiffer material, requiring more force to deform the material a set amount. A material with a higher storage modulus relative to the loss modulus is more elastic and will therefore recover more than a material with a closer ratio; the ratio of the loss (G′) to storage (G′) modulus is reported as tan δ. A purely elastic material would have a tan δ=0, while a purely viscous material would have tan δ=∞. The comparison of these parameters at two frequencies is shown in Table 1.

Consistent with the discussion above, there is a large difference in modulus between samples 11010-1 and 11010-2, but only a modest frequency dependence in either sample. Also, the tan δ values are quite close between the two samples, indicating a similar relative level of elasticity, albeit requiring different levels of force to achieve the same degree of deformation. Both samples show a modest decrease in tan δ, indicating both materials become slightly more elastic with increasing frequency. It should be noted that this test subjects the sample to small amplitudes of deformation; larger degrees of deformation could require different levels of force, hence resulting in a different modulus, but the test implicitly assumes that the experiment is performed in the linear viscoelastic regime of the material.

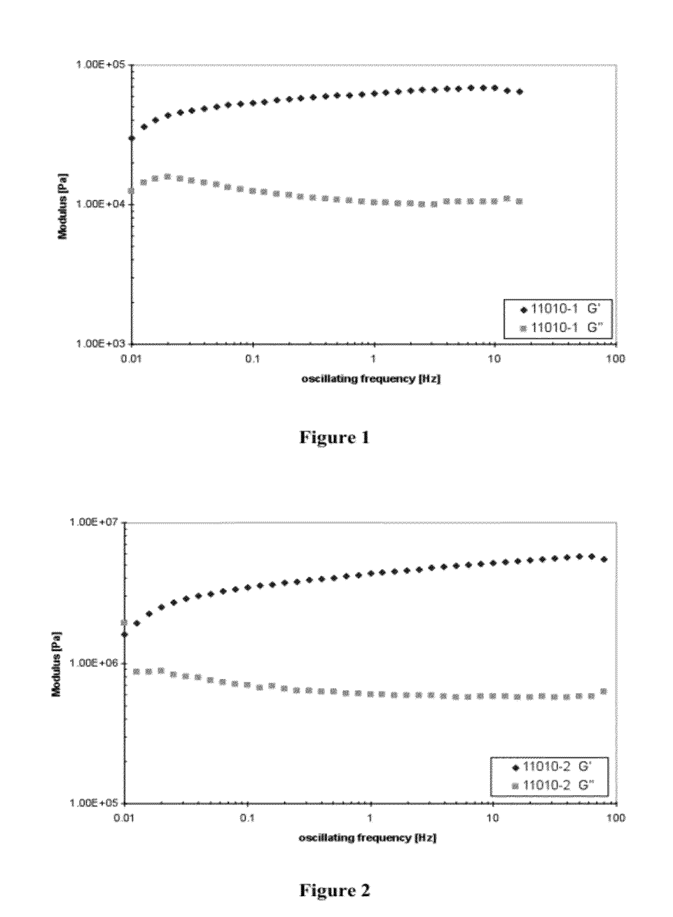

With regard to samples 11010-1 and 11010-2, the storage modulus G′ and the loss modulus G″ are plotted in FIG. 1 and FIG. 2, respectively, as a function of frequency (“Frequency Sweep”). The values of G′, G″ and G* at 0.1 Hz and at 1.0 Hz are provided in Table 1 hereinbelow.

TABLE 1

| | Frequency | | | | |
|---|---|---|---|---|---|
| Sample | [Hz] | G′ [Pa] | G″ [Pa] | G* [Pa] | tan delta (δ) |
| 11010-1 | 0.1 | 5.37E+04 | 1.25E+04 | 5.51E+04 | 0.23 |
| | 1.0 | 6.26E+04 | 1.03E+04 | 6.34E+04 | 0.17 |
| 11010-2 | 0.1 | 3.45E+06 | 6.92E+05 | 3.52E+06 | 0.20 |
| | 1.0 | 4.32E+06 | 5.99E+05 | 4.36E+06 | 0.14 |

### Example 11

A putty formulation was prepared according to the procedure provided in Example 1. The putty contained 42.5% water, 3.3% bentonite, 13.8% jojoba oil, 39.9% flour, and 0.5% tetrasilver tetroxide. The putty was soft and slightly sticky, relative to the formulation of Example 6, but exhibited both high pliability and excellent phase stability.

### Example 12

The putty formulation of Example 11 was subjected to rheological evaluation using a TA Instruments ARG2 rheometer.

Small amplitude oscillatory rheometry was conducted on the provided samples using a TA Instruments ARG2 rheometer. A two-centimeter, stainless steel parallel plate geometry was used to prevent the bridging effects that can occur in cone geometries when particle sizes might be significant. A gap of 1000 microns was used.

The samples were initially subjected to a torque sweep at 1 Hz to identify the suitable oscillating torque for the frequency sweep based on waveform shape and the onset of non-linear viscoelastic behavior. Based on this work, a torque of 1000 μN-m was determined.

A frequency sweep was then conducted on the sample, from 0.01 to 100 Hz at 25° C., using the oscillating torque amplitudes described above. Ten points were collected per decade of frequency.

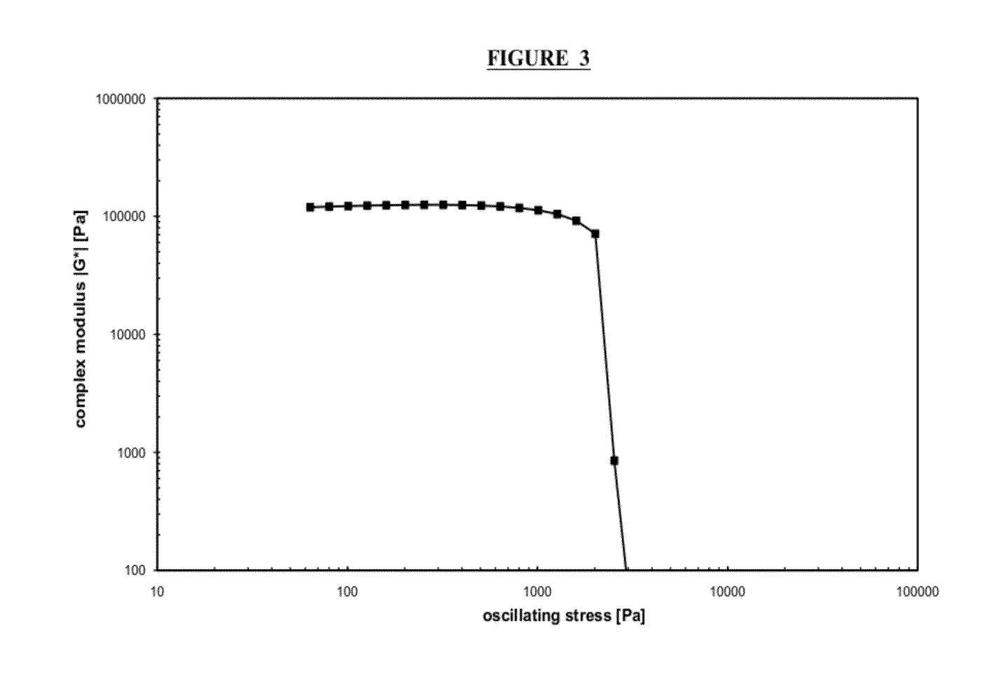

The results from the torque sweep are shown in FIG. 3. The sample showed non-linear behavior at approximately 2,000 Pa.

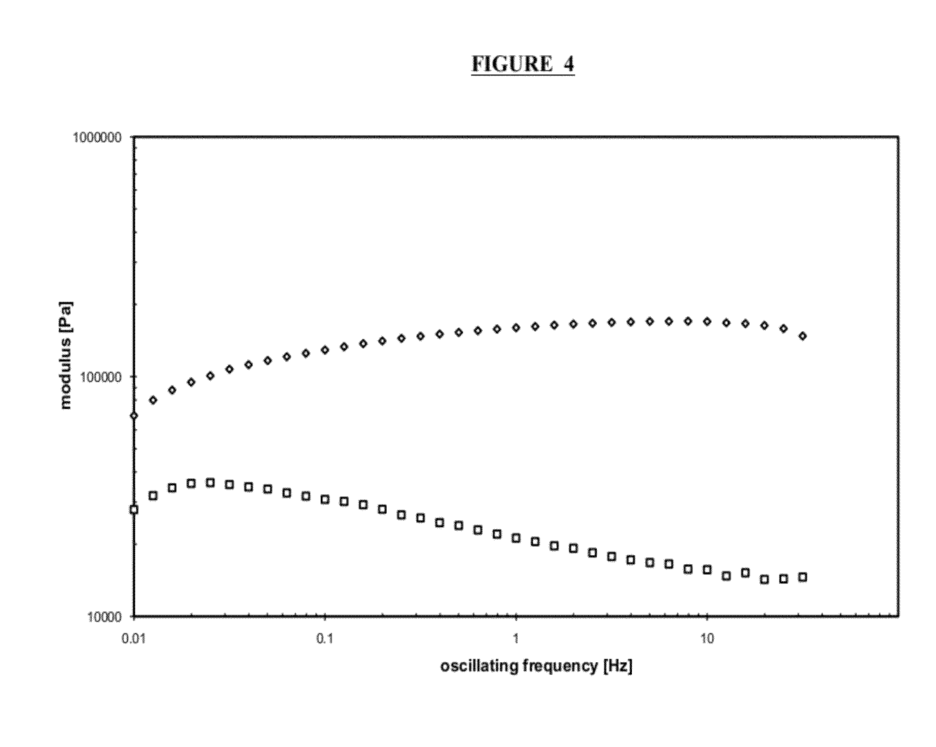

The frequency sweep data are plotted in FIG. 4. The sample exhibited a fairly flat modulus behavior, indicating little dependency on frequency.

This data is summarized at 3 frequencies (0.1 Hz, 1.0 Hz, and 10 Hz) in Table 2. The behavior of the sample was predominantly elastic, with the complex modulus ranging from about 1×10⁵ to 2×10⁵ Pa.

TABLE 2

| | Frequency | | | | |
|---|---|---|---|---|---|
| Sample | [Hz] | G′ [Pa] | G″ [Pa] | G* [Pa] | tan delta (δ) |
| 11264-2 | 0.1 | 1.29E+05 | 3.08E+04 | 1.33E+05 | 0.24 |
| | 1.0 | 1.60E+05 | 2.12E+04 | 1.61E+05 | 0.13 |
| | 10 | 1.70E+05 | 1.57E+04 | 1.71E+05 | 0.09 |

### Example 13

A formulation was prepared according to the procedure provided in Example 1, containing 69.8% water, 9.3% bentonite, 8.1% jojoba oil, 9.3% flour, 3% zinc oxide, and 0.5% tetrasilver tetroxide. The formulation was soft, having a soft putty or plaster-like consistency, and exhibited both high pliability and excellent phase stability.

### Example 14

A formulation was prepared according to the procedure provided in Example 1, containing 68.5% water, 18.3% bentonite, 7.9% jojoba oil, 4.8% zinc oxide, and 0.5% tetrasilver tetroxide. The formulation was soft, having a plaster-like consistency and exhibited both high pliability and excellent phase stability.

### Example 15

The formulation of Example 13 was prepared according to the general procedure provided in Example 1, however, the mixing of the bentonite into the water was conducted for about 10 minutes. Despite having a composition substantially identical to that of Example 13, the formulation failed to develop the requisite viscosity or body. The formulation had a paste-like consistency, even after additional mixing time was provided after the silver oxide and zinc oxide were introduced.

### Example 16

A formulation was prepared according to the procedure provided in Example 1, containing 65.4% water, 11.3% bentonite, 17.8% jojoba oil, 5% zinc oxide, and 0.5% tetrasilver tetroxide. The formulation was soft, exhibiting a plaster-like consistency and exhibited both high pliability and excellent phase stability.

### Example 17

A putty formulation was prepared according to the procedure provided in Example 1, containing 40.6% water, 3.4% bentonite, 14.2% jojoba oil, 40.5% wheat flour, 0.5% tetrasilver tetroxide, 0.5% Allantoin, 0.1% Benzathonium Cl, and 0.5% Lidocaine.

### Example 18

A putty formulation was prepared according to the procedure provided in Example 1, containing 39.0% water, 3.1% bentonite, 13.5% jojoba oil, 40.0% wheat flour, 0.5% tetrasilver tetroxide, 1.0% Clotrimazole, 5% salicyclic acid, and 0.1% colloidal oatmeal.

### Example 19

The anti-microbial efficacy of various formulations was tested and compared using a Kirby-Bauer type test, as follows:

Ready-made Muller-Hilton agar was streaked with the bacterial inoculum using a sterile applicator. The sample was allowed to sit for 5 minutes to ensure that the bacteria adhere to the surface of the agar. Subsequently, an antibiotic sterile blank disc was pressed against a known quantity of the formulation being tested. Multiple duplicate discs were used to verify the data. The disc was pressed against the surface of the agar, making sure not to damage the disc or the agar. Each agar plate was then inverted and allowed to sit in the incubator at 37° C. for 24 hours. The plates were subsequently removed from the incubator, and the zone of inhibition was measured using a ruler.

The anti-microbial efficacy of eight formulations was tested and compared using the procedure detailed above, using Enterococcus faecalis.

Formulation Nos. 1-7 correspond to the formulations produced in Example Nos. 6, 11, 13, 14, 16, 17, and 18. Formulation No. 8 was a control formulation, produced according to the procedure outlined in Example 1. The control formulation was a putty containing: 40% water, 3.4% bentonite, 14.2% jojoba oil, and 40% wheat flour. No antibiotic was included in the control formulation.

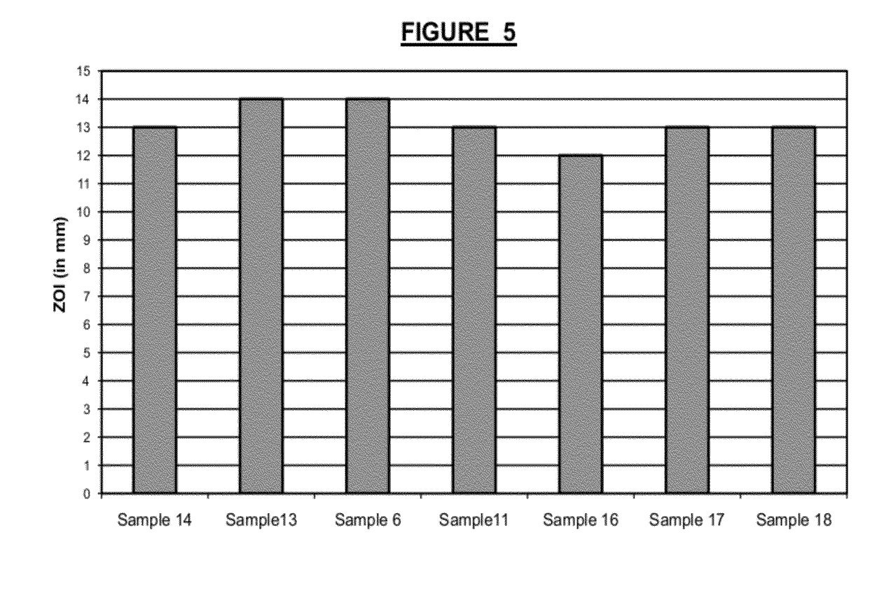

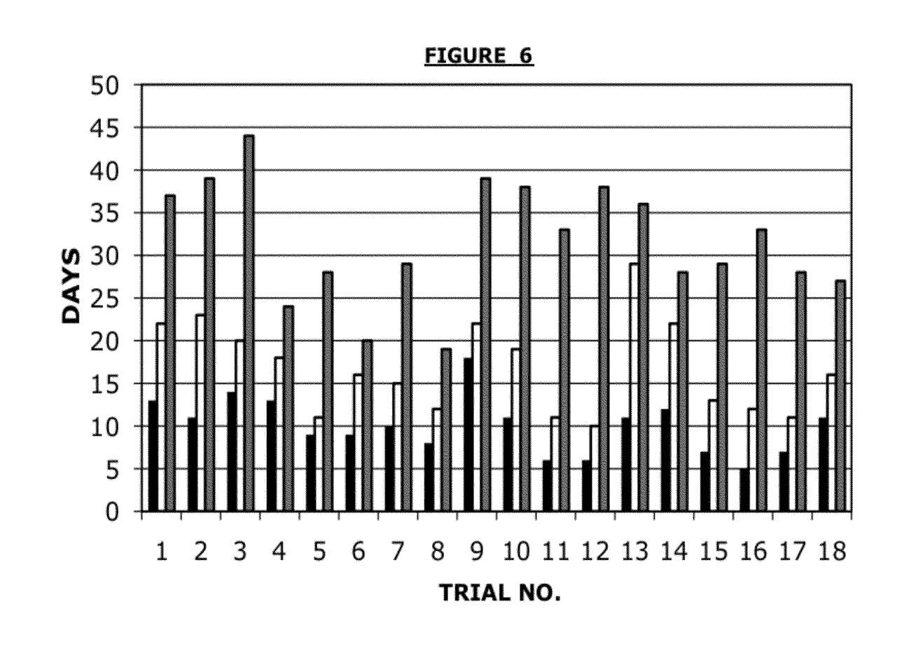

The zone of inhibition for the control formulation was substantially 0 mm. By sharp contrast, the zone of inhibitions for Formulation Nos. 1-7 all fell within a narrow range of about 12-14 mm (see FIG. 5). Such a large zone of inhibition may be considered a clear manifestation of the appreciable antibiotic activity of the inventive formulations, and was obtained using a low concentration of the silver oxide. Moreover, the large zone of inhibition may be especially noteworthy in view of the extremely high viscosities exhibited by the inventive putty formulations.

### Examples 20-26

Formulations having compositions generally along the lines of Example 11, were prepared according to the procedure provided in Example 1. In Example 20, the putty contained 42.5% water, 3.3% bentonite, 13.8% jojoba oil, 39.9% flour, and 0.5% tetrasilver tetroxide, as in Example 11. The flour was a whole wheat flour. The putty was soft and slightly sticky, relative to the formulation of Example 6, but exhibited both high pliability and moldability, and excellent phase stability.

In Example 21, rice bran flour replaced the wheat flour, and the silver(II) oxide concentration was increased to 4%. To obtain a similar consistency as that obtained in Example 20, the ratio of filler (rice flour) to water was increased from 0.94 to 1.48, representing a 58% increase in filler, relative to the wheat flour of Example 20. The putty had a dark gray color, which may largely be due to the relatively high concentration of the silver(II) oxide. The putty was soft and slightly sticky, relative to the formulation of Example 6, and exhibited excellent phase stability. The formulation had a somewhat grainy appearance and was moldable, though less so than the putty of Example 20.

In Example 22, corn starch replaced the wheat flour of Example 20, while the silver(II) oxide concentration was maintained at 0.5%. To obtain a similar consistency as that obtained in Example 20, the ratio of filler (corn starch) to water was increased from 0.94 to 1.09, representing a 17% increase in filler, relative to the wheat flour of Example 20. The putty had a substantially white appearance. The putty was soft and slightly sticky, relative to the formulation of Example 6, and exhibited excellent phase stability. The formulation was moldable, though less so than the putty of Example 20.

In Example 23, potato starch replaced the wheat flour of Example 20, while the silver(II) oxide concentration was increased at 1.5%. To obtain a similar consistency as that obtained in Example 20, the ratio of filler (potato starch) to water was increased from 0.94 to 1.25, representing a 33% increase in filler, relative to the wheat flour of Example 20. The putty had a yellow tinge. The putty was soft and slightly sticky, relative to the formulation of Example 6, and exhibited excellent phase stability. The formulation was more grainy than the putty of Example 22 and was moldable, though less so than the putty of Example 20.

In Example 24, the whole wheat flour of Example 20 was used, but the silver(II) oxide was replaced by silver(I) oxide (0.5%). The appearance of the putty, consistency, and phase stability appeared to be identical, or substantially identical to those of the putty of Example 20.

In Examples 25 and 26, the putty formulation of Example 20 was prepared, again, according to the general procedure of Example 1. In each formulation, a different topical antibiotic material was used instead of the silver(II) oxide. The concentration of each antibiotic material was selected according to the concentration of the antibiotic material in commercially available ointments. Thus, in Example 25, the antibiotic material was clotrimazole, 1% by weight; in Example 26, the antibiotic material was erythromycin, 2% by weight. The appearance, consistency, and phase stability of the putties of Example 25 appeared to be identical, or substantially identical to those of the putty of Example 20.

### Example 27

Fifty four patients were treated at Irvine3 Circulation/Vascular Labs (Chieti-Pescara University, Pescara, Italy). Patients were matched with other patients of similar age and similar general health condition, and having complex ulcers of similar type, size and severity. Effectively, 18 groups of three patients were formed for the purpose of comparative testing.

Within each group of three, a first patient was treated with conventional cleaning and compression management methods. A second patient within each group was treated with an ointment, containing approximately 0.9% silver(II) oxide and 6.8% ZnO in a beeswax and jojoba oil base. The ointment was applied around and at the edge of the ulcerated areas and on the ulceration, following the identical conventional cleaning methods used on the first patient.

The third patient within each group was treated with an antibiotic-containing putty of the present invention. The antibiotic, consisting essentially of tetrasilver tetroxide (silver(II) oxide), was dispersed within the putty, which had the composition of the putty described in Example 6, and was prepared according to the procedure provided in Example 1.

FIG. 6 provides bar graphs showing the wound closure data from each of the 18 comparative clinical trials. In FIG. 6, associated with each trial number are three juxtaposed bar graphs, the right-most of which represents the patient subjected to the conventional treatment, the middle bar graph represents the patient treated with the ointment containing the silver(II) oxide, and the left-most of which represents the patient treated using the formulation of the present invention.

On average, the complex ulcers treated by conventional means required over 31 days to close, on average. The complex ulcers treated with the silver(II) oxide based ointment required almost 17 days to close, on average, an appreciable improvement over the results for the control group. The complex ulcers treated with the antibiotic-containing putty of the present invention closed after just over 10.1 days, on average, about ⅓ of the time required for the wounds of the control group, and about 40% less time with respect to the excellent result achieved using the ointment. The performance of the inventive antibiotic putty is more surprising in view of the relatively low concentration of antibiotic (0.5% silver(II) oxide) in the putty, with respect to the concentration of the same antibiotic (˜0.9% silver(II) oxide) in the antibiotic ointment formulation. The improved performance is even more surprising in view of past experience showing that for a given concentration of antibiotic material, more viscous formulations may considerably less efficacious from a bacteriocidal standpoint.

As used herein in the specification and in the claims section that follows, the term “percent”, or “%”, refers to percent by weight, unless specifically indicated otherwise.

Similarly, the term “ratio”, as used herein in the specification and in the claims section that follows, refers to a weight ratio, unless specifically indicated otherwise.

As used herein in the specification and in the claims section that follows, the term “silver (II) oxide” refers to a silver oxide whose unit structure contains silver and oxygen in a substantially 1:1 molar ratio. The term “silver (II) oxide” is specifically meant to include Ag₄O₄ (often represented as Ag₂O₃.Ag₂O) and AgO.

As used herein in the specification and in the claims section that follows, the term “silver (I) oxide” refers to a silver oxide whose unit structure contains silver and oxygen in a substantially 2:1 molar ratio. The term “silver (I) oxide” is specifically meant to include Ag₂O.

As used herein in the specification and in the claims section that follows, the term “antibiotic” refers to a substance that selectively attacks and destroys at least one species or type of microorganism, while exhibiting relative inertness with respect to human and/or mammalian cells. More typically the antibiotic substance selectively attacks and destroys at least one species or type of microorganism that commonly populates the skin, surface wounds, bedsores and the like, while exhibiting relative inertness, with respect to skin cells of humans and/or mammals. The term “antibiotic” is specifically meant to exclude anti-microbial preservatives, both anti-fungal preservatives and anti-bacterial preservatives. Such anti-fungal preservatives include, but are not limited to, compounds such as benzoic and ascorbic acids and salts thereof, and phenolic compounds such as methyl, ethyl, propyl and butyl p-hydroxybenzoate (parabens). Antibacterial preservatives include, but are not limited to, compounds such as quaternary ammonium salts, alcohols, phenols, mercurials and biguanidines. The term “antibiotic” is specifically meant to exclude anti-microbial preservatives such as table salt and the like, vinegar, sodium nitrate, sodium nitrite, and sulfites. The term “antibiotic” is specifically meant to include, without being limited to, silver oxides such as silver(I) oxide and silver(II) oxide, silver sulfadiazine, and any other topical antibiotics that are efficacious in the treatment of serious skin wounds such as bedsores, skin ulcers, and puncture wounds, or that are efficacious in the treatment of mundane skin wounds. The term “antibiotic” is specifically meant to include “classic” topical antibiotics such as Bacitracin, Neomycin, Erythromycin and Chloramphenicol. Additional topical antibiotic substances may be readily apparent to those of ordinary skill in the art.

As used herein in the specification and in the claims section that follows, the term “therapeutically effective amount”, with respect to an antibiotic substance or formulation, refers to a quantity that produces a positive result in the treatment of at least one topical infection.

As used herein in the specification and in the claims section that follows, the term “therapeutically effective concentration”, with respect to an antibiotic substance within a formulation or medical device, refers to a concentration of the antibiotic, within the formulation or medical device, which produces a positive result in the treatment of at least one topical infection.

As used herein in the specification and in the claims section that follows, the term “putty”, with respect to a substance or formulation, is meant to refer solely to the physical consistency of the substance or formulation.

As used herein in the specification and in the claims section that follows, the term “plaster”, with respect to a substance or formulation, is meant to refer solely to the physical consistency of the substance or formulation.

As used herein in the specification and in the claims section that follows, the term “largely includes”, with respect to a component within a formulation, refers to a content of at least 30%, by weight.

As used herein in the specification and in the claims section that follows, the term “mainly includes”, with respect to a component within a formulation, refers to a content of at least 50%, by weight.

As used herein in the specification and in the claims section that follows, the term “predominantly includes”, with respect to a component within a formulation, refers to a content of at least 65%, by weight.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A solid biocompatible formulation suitable for insertion within chronic and acute wounds of humans and animals, the formulation comprising a topical antibiotic, a biocompatible humectant, a biocompatible viscosity-building agent, and at least 5% water, by weight, said humectant and said viscosity-building agent intimately mixed within the formulation,
the formulation formulated and adapted whereby the formulation remains a solid over at least an entire temperature range of 20° C. to 35° C.,
the solid formulation having a storage modulus (G′) and a loss modulus (G″), both measured at 25° C. and within a frequency range of 0.1 Hz to 1.0 Hz, and a complex modulus (G*), defined by:
G*=(G′²+G″²)^{1/2}
the formulation having at least one of the following five rheological properties:
(1) in a torque sweep at a frequency of 1.0 Hz, said complex modulus achieves a plateau or a maximum of at least 4.0×10⁴ Pa, at least 6.0×10⁴ Pa, at least 8.0×10⁴ Pa, or at least 10.0×10⁴ Pa;
(2) in said torque sweep, said complex modulus drops sharply, or begins to exhibit non-linear behavior, at an oscillating stress of at least 800 Pa, at least 900 Pa, at least 1000 Pa, at least 1200 Pa, at least 1500 Pa, or at least 2000 Pa;
within said frequency range, at least one point:
(3) said storage modulus is at least 1.0×10⁴ Pa, at least 2.0×10⁴ Pa, at least 3.0×10⁴ Pa, at least 4.0×10⁴ Pa, at least 5.0×10⁴ Pa, or at least 6.0×10⁴ Pa;
(4) said loss modulus is at least 0.4×10⁴ Pa, at least 0.5×10⁴ Pa, at least 0.6×10⁴ Pa, at least 0.8×10⁴ Pa, at least 1.0×10⁴ Pa, at least 1.5×10⁴ Pa or at least 2.0×10⁴ Pa;
(5) said complex modulus is at least 1.05×10⁴ Pa, at least 1.05×10⁴ Pa, at least 2×10⁴ Pa, at least 3.0×10⁴ Pa, at least 4.0×10⁴ Pa, or at least 6.0×10⁴ Pa.

2. The formulation of claim 1, wherein in said torque sweep, said complex modulus achieves said plateau or said maximum of at least 4.0×10⁴ Pa, at least 6.0×10⁴ Pa, at least 8.0×10⁴ Pa, or at least 10.0×10⁴ Pa.

3. The formulation of claim 2, wherein in said torque sweep, said complex modulus drops sharply, or begins to exhibit said non-linear behavior, when said oscillating stress is at least 800 Pa, at least 900 Pa, at least 1000 Pa, at least 1200 Pa, at least 1500 Pa, or at least 2000 Pa.

4. The formulation of claim 1, wherein, at least one point within said frequency range, said storage modulus is at least 5.0×10⁴ Pa, or at least 6.0×10⁴ Pa, and said loss modulus is at least 0.8×10⁴ Pa, at least 1.0×10⁴ Pa, at least 1.5×10⁴ Pa, or at least 2.0×10⁴ Pa.

5. The formulation of claim 1, wherein substantially throughout said frequency range, said storage modulus is at least 3.0×10⁴ Pa, at least 4.0×10⁴ Pa, at least 5.0×10⁴ Pa, or at least 6.0×10⁴ Pa.

6. The formulation of claim 1, wherein substantially throughout said frequency range, said loss modulus is at least 0.6×10⁴ Pa, at least 0.8×10⁴ Pa, at least 1.0×10⁴ Pa, at least 1.5×10⁴ Pa, or at least 2.0×10⁴ Pa.

7. The formulation of any one of claims of claim 1, wherein a water concentration within the formulation is at least 7%, at least 10%, at least 20%, at least 30%, at least 40%, at least 50%, or at least 60%.

8. The formulation of claim 1, wherein said antibiotic is selected from the group of topical antibiotics consisting of silver(II) oxide, silver(I) oxide, silver sulfadiazine, Bacitracin, Neomycin, Erythromycin and Chloramphenicol.

9. The formulation of claim 1, wherein said antibiotic consists largely of, predominantly of, or substantially of, silver oxide.

10. The formulation of claim 1, wherein the formulation is a putty throughout said temperature range.

11. The formulation of claim 1, the formulation including at least 4%, at least 7%, at least 12%, at least 20%, or at least 30% of said humectant.

12. The formulation of claim 1, wherein said humectant includes, largely includes, predominantly includes, or consists essentially of a liquid wax ester.

13. The formulation of claim 1, further comprising an absorbefacient.

14. The formulation of claim 1, further comprising an absorbefacient, wherein a combined weight content of said viscosity-building agent and said absorbefacient within the formulation is at least about 4%, at least 6%, at least 8%, at least 10%, or at least 15%.

15. The formulation of claim 1, wherein said viscosity-building agent includes, largely includes, or consists essentially of at least one of a hydrophyllic clay, a flour, and a starch.

16. The formulation of claim 15, wherein said hydrophyllic clay is selected from at least one of the group of hydrophyllic clays consisting of a smectite, sepiolite, and palygorskite.

17. The formulation of claim 14, wherein a weight ratio of said at least one viscosity-building agent and said absorbefacient to said humectant is at least 0.25:1, at least 0.4:1, at least 0.6:1, or at least 1:1.

18. The formulation of claim 1, adapted whereby a plug or piece of the formulation is insertable into a wound cavity in an integral fashion.

19. The formulation of claim 1, adapted whereby a plug or piece of the formulation is removable from a wound cavity in an integral fashion.

20. The formulation of claim 1, adapted whereby a plug or piece of the formulation provides a gentle pressure against a surface within a wound cavity.
